(19) United States
(12) Patent Application Publication
(10) Pub. No.: US 2009/0214638 A1
(43) Pub. Date: Aug. 27, 2009

(54) LIPOSOME ENCAPSULATED POLY-ICLC METHOD TO PROPHYLACTICALLY TREAT AN AVIAN INFLUENZA VIRAL INFECTION

(76) Inventor: Jonathan WONG, Medicine Hat (CA)

Correspondence Address: CERMAK KENEALY VAIDYA & NAKAJIMA LLP, 515 E. BRADDOCK RD, ALEXANDRIA, VA, 22314, US

(21) Appl. No.: 12/433,961

(22) Filed: May 1, 2009

Related U.S. Application Data
(63) Continuation of application No. PCT/CA2007/001928, filed on Oct. 30, 2007.
(60) Provisional application No. 60/856,310, filed on Nov. 3, 2006.

(51) Int. Cl. A61K 9/127; A61K 31/713

(52) U.S. Cl. 424/450

(57) ABSTRACT

A method of treating an avian influenza viral infection using a poly ICLC formulation with improved therapeutic efficacy is disclosed. The poly ICLC is encapsulated within liposomes which provides a drug delivery system with slow sustained release characteristic and which has the ability to target the drug to sites of viral infection without causing systemic burden to normal tissues, thereby enhancing the immunological and biological activities of poly ICLC. This poly ICLC formulation has been found to be particularly effective in treating an avian influenza viral infection and in particular, an avian H5N1 influenza viral infection.

### BACKGROUND OF THE INVENTION

This application is a continuation under 35 U.S.C. § 120 of PCT/CA2007/001928, filed Oct. 30, 2007, and which claims priority under 35 U.S.C. § 119(e) to U.S. Provisional Patent Application No. 60/856,310 filed on Nov. 3, 2006, which are incorporated in their entireties by reference.

### FIELD OF THE INVENTION

The present invention relates to a method of treating an avian influenza viral infection using a poly ICLC formulation with improved therapeutic efficacy.

### BACKGROUND OF THE INVENTION

There is currently a concern that the world is moving closer to a global influenza pandemic since the cumulative number of confirmed human cases of Avian Influenza A (H5N1) reported to the World Health Organization has increased every year since 2003 (from 4 cases in 2003 to 109 cases in 2006).

With both seasonal and avian influenza virus developing increasing resistance to both antiviral drugs and vaccines, and in view of the difficulties in vaccine design and production due to the constant structural changes in the virus, there are compelling reasons to develop novel antiviral approaches that are broad-spectrum and independent of the genetic make up of the influenza viruses. The development of such approaches not only will decrease the likelihood of drug resistance, but can also offer protection against new variants of influenza viruses of zoonotic origin.

The significant difference between avian or a pandemic influenza and seasonal influenza viruses are that the former virus causes massive inflammation in the respiratory tract of the infected individuals. The inflammation of the respiratory tract caused by avian and pandemic influenza viruses are estimated to be 10 times higher than the level of that caused by normal seasonal influenza, and is a contributing factor to the increased fatality seen in both bird flu and pandemic flu victims. The development of novel methods to mitigate the massive inflammation is an important but sometimes overlooked part of comprehensive strategy against avian and pandemic flu.

Scientists have found that double-stranded RNAs (dsRNAs) are very potent biologic modifiers and can potentially serve as a broad-spectrum immunoprophylaxis against avian influenza infection. dsRNAs can exert a profound influence on cells at nanomolar concentrations. The modulating effects of dsRNA include a broad spectrum of actions at the molecular and cellular levels. At the molecular level, dsRNAs can elicit biological effects such as interferon synthesis, induction of protein kinase, induction of 2-5 A polymerase, enhancement of histocompatibility antigen and inhibition of metabolism. And at the cellular level, dsRNA can elicit biological effects such as pyrogenicity, mitogenicity, macrophage activation, activation of cell-mediated immunity and induction of antiviral state. One particular characteristic of dsRNAs is its immunomodulating effect in antimicrobial and anticancer therapies. In particular, the double-stranded RNA poly ICLC, or PICLC for short, was found highly effective as an antiviral or antitumor agent. It is believed that the double-stranded RNA poly ICLC can be used to attenuate the host to elicit the beneficial components of the antiviral pathways (e.g. the 2′-5′ oligoadenylate synthase pathway) while downregulate the damaging effects of the pro-inflammatory cytokine pathways.

Poly ICLC is a synthetic dsRNA consisting of polyriboinosinic and polyribocytidylic acid strands (poly I.poly C) stabilized with poly-L-lysine and carboxymethylcellulose. The resulting poly ICLC is thermodynamically more stable than poly I.poly C. Poly ICLC has been shown in clinical trials to be effective in the cancer treatment of gliomas (Salazar, A, M. & al., Neurosurgery 38:1096-1104). It has also been shown in a number of studies to be effective in the immunotherapy of viral infection including influenza (Wong, J. P. Antimicrob. Agents Chemother, 39:574-2576), rabies (Baer, G. M., J. Infect. Dis. 136:286-292). Rift Valley fever (Kende, M., J. Biol. Response Modifiers 4:503-511) and Venequelan equine encephamyelitis (Stephen, E. L., J. Infect. Dis. 136:267-272).

Although poly ICLC is a promising immunomodulator which has great potential in antimicrobial and anticancer therapies, it has been shown to produce serious side effects in humans, especially when the drug is administered in multiple high doses. Some of the reported side effects (Levine, A. S., Cancer Treat. Rep. 62:1907-1913) include fever, hypotension, leukopenia, myalgia, thrombocytopenia and poly arthalgia. The inherent toxicity problem must be overcome to render poly ICLC safer for use in humans. Furthermore, the therapeutic efficacy of poly ICLC is limited by its stability in vivo. As a ribonucleic acid, poly ICLC is susceptible to degradation in the body by serum RNAse. Although the extent of RNAse degradation of poly ICLC is much improved as compare to that of poly I poly C, the protection is not complete and poly-L-lysine and carboxymethylcellulose themselves may be susceptible to enzymatic degradation and immunological clearance in vivo. Therefore, a need exists for an improved formulation of poly ICLC which has improved therapeutic efficacy and will be safer for use in humans. Also, the therapeutic efficacy of poly ICLC against the avian influenza virus also needs to be established.

### SUMMARY OF THE INVENTION

It is an object of the present invention to provide a poly ICLC formulation having enhanced therapeutic efficacy against the avian influenza virus while reducing it toxic effect in humans.

In accordance with one aspect of the present invention, there is provided a method of prophylactically treating an avian influenza viral infection in a mammal comprising administering to the mammal an immunomodulating agent comprising poly ICLC encapsulated within liposomes.

Preferably, the liposomes used are unilamellar or multilamellar and contain at least one cationic phospholipid such as stearylamine, 1,2-diacyl-3-trimethylammonium-propane (TAP) or 1,2-triacyl-3-dimethylammonium-propane (DAP). Most preferably, the liposomes are unilamellar or multilamellar liposomes prepared from the lipids phosphatidylcholine and stearylamine, and the steroid cholesterol at a molar ratio of approximately 9:1:1, respectively. The surface liposomes may be coated with polyethylene glycol to prolong the circulating half-life of the liposomes, and with antibody for targeting to specific sites in the body.

Neutrally charged liposomes can also be used for liposomal entrapment of poly ICLC. Such neutrally charged liposomes can be prepared by using, for example phosphatidylcholine and cholesterol.

The avian influenza viruses encompassed by the present invention include all subtypes of influenza A viruses which can infect birds. There are 16 known HA subtypes and 9 known NA subtypes. Three prominent subtypes of the avian influenza A viruses that are known to infect both birds and people are influenza A H5 (nine potential subtypes of H5 are known), influenza A H7 (nine potential subtypes of H7 are known) and influenza A H9 (nine potential subtypes of H9 are known). Of these, the avian H5N1 influenza virus is preferred.

In accordance with another aspect of the present invention there is provided a method for preparing liposomal poly ICLC comprising the step of freeze-drying a mixture of liposomes and poly ICLC. Conveniently, the method includes removing organic solvent from a mixture of phospholipids, rehydrating the resulting lipids mixture with an aqueous buffer containing poly ICLC, freeze-drying the resulting lipid-poly ICLC mixture, reconstituting the resulting dried mixture, and resuspending the resulting liposome pellets with a buffer solution to the desired drug concentration prior to use. Suitable buffer can be phosphate buffered saline, normal saline or deionized water. It is important for the preparation of buffer solution to use RNAse-free water so that enzymatic degradation of poly ICLC can be minimized.

Alternate methods of preparation of liposomes include detergent dialysis, extrusion, reverse-phase evaporation (REV) and sonication. The loading of poly ICLC into the liposomes can be achieved by passive trapping and by active process such as remote loading. The unentrapped poly ICLC can be removed by centrifugation, column separation or by dialysis.

The advantages of encapsulating poly ICLC in liposomes are that the toxicity of poly ICLC is decreased, and at the same time the therapeutic efficacy of poly ICLC is increased. Furthermore, liposomal poly ICLC protects the poly ICLC from RNAse degradation in the body, thereby enhancing the immunological and biological activities of poly ICLC.

### BRIEF DESCRIPTION OF THE DRAWINGS

FIG. 1 is a graph showing results of tests relating to the therapeutic efficacy of free poly ICLC versus that of liposomal poly ICLC.

FIG. 2 is a graph showing the results of tests relating to the toxicity of free poly ICLC versus that of liposomal poly ICLC.

FIG. 3 is a graph showing results of tests relating to the therapeutic efficacy of liposomal poly ICLC versus control against low virus challenge dose of avian H5N1 influenza virus in mice.

FIG. 4 is a graph showing results of tests relating to the therapeutic efficacy of liposomal poly ICLC versus control against high virus challenge dose of avian H5N1 influenza virus in mice.

### DETAILED DESCRIPTION OF THE PREFERRED EMBODIMENTS

Poly ICLC

Poly ICLC was prepared by the Pharmaceutical Services, College of Pharmacy University Of Iowa (Iowa City, Iowa), and was provided by the National Institute of Health (Bethesda, Md.). Each milliliter of poly ICLC contained 2 mg poly I.poly C, 1.5 mg poly-L-lysine, and 5 mg carboxymethylcellulose in 0.9% sodium chloride.

Encapsulated-Liposome Poly ICLC

Liposomes are microscopic lipid vesicles consisting of one or more lipid bilayer(s) and aqueous compartment(s). The primary constituents of liposomes are usually a combination of phospholipids and steroid, such as cholesterol. The phospholipids can be positively, neutrally and negatively charged. Liposomes made from positively and negatively charged phospholipids are called cationic and anionic liposomes, respectively. DNA and RNA are usually negatively charged, therefore, cationic liposomes are the liposomes of choice for making liposomal poly ICLC formulation. The cationic phospholipid used for making liposomal poly ICLC is preferably stearylamine, 1,2--diacyl-3-trimethylammonium-propane (TAP) or 1,2--triacyl-3-dimethylammonium-propane (DAP). Cholesterol is included for stabilization of the bilayer. The surface liposomes may be coated with polyethylene glycol to prolong circulation thereof. Proteins can also be combined with the liposome membranes to promote binding with specific cell receptors.

Liposomes used for entrapment of poly ICLC may be large multilamellar vesicles (MLVs), small unilamellar vesicles (SUVs) or large unilamellar vesicles (LUVs). Preferably, MLVs are used for preparing liposomal poly ICLC.

When used as a drug delivery system, liposomes are known to have a slow sustained release characteristic and the ability to target drugs to sites of infection and tumor without causing systemic burden to normal tissues. Liposomes have been used successfully to entrap a number of therapeutic drugs, including antibiotics, antivirals, and anticancer. Because of these attributes, liposomal poly ICLC is an excellent drug delivery system which can significantly decrease the dose-related toxicity of poly ICLC. Furthermore, liposome-encapsulation protects the poly ICLC from RNAse degradation in the body, thereby enhancing the therapeutic efficacy of poly ICLC.

Preparation

The liposomes were prepared using 210 mg of phosphatidylcholine (210 .mu.mole), 23.2 mg stearylamine (23.2 .mu.mole) and 8.1 mg cholesterol (30 .mu.mole). The lipids were added in a 100 ml round bottom flask, 2 ml of chloroform was added to dissolve the lipids. The round bottom flask was rotary evaporated in a 45.degree. C. water bath until a dried lipid film was formed. The flask was then placed in a vacuum oven (45.degree. C., −80 Kpa) for one hour to remove residual organic solvent. The lipid film was then reconstituted with 3 ml of poly ICLC (2 mg/ml) followed by 3 ml of 0.9% NaCl. Other suitable buffers can be phosphate buffered saline, normal saline or deionized water. It is important for the preparation of buffer solution to use RNAse-free water to minimize degradation of poly ICLC. The lipid-drug mixture was then transferred to a screwcapped tube, mixed well, and frozen by immersing the tube in liquid nitrogen. The sample was then lyophilized overnight until all the liquid was removed to obtain a white dried powder. Following lyophilization, the sample was rehydrated with 100-150 .mu.l 0.9% NaCl, incubated for 15 min, at 45.degree. C., and left undisturbed for 2 hr. at room temperature. The liposomal poly ICLC was diluted in sterile 0.9% NaCl and washed using an ultracentrifugation step. The liposome pellet was then resuspended with a buffer solution to the desired drug concentration for administration into mice.

The surface of the liposomes may be coated with polyethyleneglycol to prolong circulation and with an antibody to increase the affinity of the liposome to specific sites of infection and tumor.

Neutrally charged liposomes can also be used for liposomal entrapment of poly ICLC. For example, the neutrally charged liposomes can be prepared using phosphatidylcholine and cholesterol.

Other methods of preparation to produce liposomes include detergent dialysis, extrusion, reverse-phase evaporation (REV) and sonication. The loading of poly ICLC into the liposomes can be achieved by passive trapping or by active process, such as remote loading. The unentrapped poly ICLC can be removed by centrifugation, column separation or by dialysis.

Adaptation of Egg-Propagated Influenza A/PR/8 Virus in Mice

Using conventional procedures, influenza A/PR/8 virus was communicated to mice through lung passages by four blind passages utilizing egg-propagated virus (available from ATTC, Parklawn, Md.) as the initial inoculum. The virus became pathogenic in mice as early as the third passage. The symptoms of influenza were standing fur, rapid loss of body weight, grouping together and significant loss of animal's movement inside the cages. Post-mortem examination of the infected mice revealed severe pulmonary lesions and pulmonary enlargement was also observed in some mice.

Testing

Liposome-encapsulated poly ICLC was administered to the mice by intranasal, intraperitoneal or intravenous routes. The volumes of inoculum used were 50 .mu.l for the intranasal route and 100 .mu.l by the intraperitoneal and intravenous routes. For the intranasal and intraperitoneal routes, mice were anaesthetized with sodium pentobarbital prior to administration of the drug. When the animals were unconscious, they were carefully supported by hands with their nose up, and the antiviral agents were gently applied with a micropipette into the nostrils. The applied volume was naturally inhaled into the lungs.

Groups of anesthetized mice (5-10 mice per group) were given one or two doses (20 .mu.g/dose) of poly ICLC or liposome-encapsulated poly ICLC by the intraperitoneal or intravenous route. The doses were given to the mice 7, 14 and 21 days prior to virus challenge. The mice were then intranasally infected with 10 LD.sub.50 mouse-adapted influenza A/PR/8 virus. At day 14 post virus infection, the number of mice which survived the virus challenged was recorded.

Results

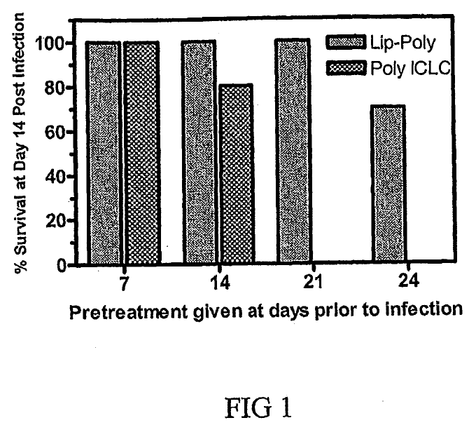

The efficacy of free and liposome-encapsulated poly ICLC for the prophylactic protection of mice against lethal challenges of influenza A infection in mice is shown in FIG. 1. In comparison, mice which were administered free poly ICLC within 7 days prior to virus infection had a 100% survival rate at day 14 post virus infection. However when pretreatment of free poly ICLC were given at days 14 and 21 prior to virus challenge, the survival rates at day 14 post infection decreased. In contrast, mice which were given liposome-encapsulated poly ICLC (MLV poly ICLC) within days 7 and 14 prior to virus challenge had a 100% survival rate at day 14 post virus infection. These results showed that liposome encapsulation did not adversely affect the antiviral and immunomodulating activities of poly ICLC, but, rather enhanced these activities by prolonging the antiviral state.

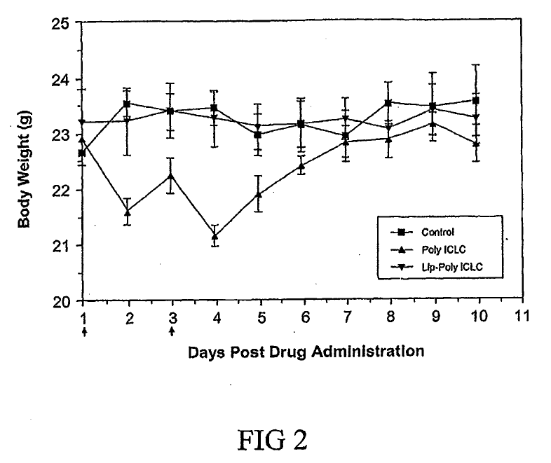

Referring now to FIG. 2, there is shown the effect of toxicity of free and liposomal poly ICLC on mice as measured by their body weight. Mice which have a toxic dose of poly ICLC will experience signs, such as rapid loss in body weight, piloerection and decreased body movement. Mice were administered two daily doses of 30 .mu.g/animal of free poly ICLC. Referring to FIG. 2, the first dose was given at day 2 post drug administration and the second dose was given at day 0 post drug administration. It was found that mice were loosing up to 2 g (close to 10% of total body weight) within 1-3 days after each administration. In addition to the loss of body weight, these mice also showed abnormal symptoms or signs of piloerection (ruffled fur) and decreased body movement. In contrast, mice given identical doses of the liposome-encapsulated poly ICLC did not have significant loss of body weight, nor did they show any signs of piloerection and loss of movement. Therefore, it was found that free unencapsulated poly ICLC had high toxicity, whereas liposome-encapsulated poly ICLC had a low toxicity as shown from the results in FIG. 2. The mice which were administered with liposomal poly ICLC did not exhibit a significant loss of body weight.

In conclusion, the results showed that free poly ICLC when administered directly into mice provided limited protection against influenza A virus infection. Moreover, repeated high doses of poly ICLC were shown to produce side toxic effects in mice. In contrast, liposome-encapsulated poly ICLC provided effective treatment against viral infections by enhancing the therapeutic efficacy while decreasing the toxicity of poly ICLC.

Liposome-Encapsulated Poly ICLC Against Avian H5N1 Influenza Viral Infection in Mice

To demonstrate the prophylactic effect of liposomal poly ICLC against avian influenza H5N1 virus, a lethal respiratory murine model was established using a wild type of H5N1 influenza A virus isolated from infected chickens. The establishment of such an infection model for H5N1 influenza virus of avian origin in mice was pivotal in permitting efficacy determination of the antiviral activity of liposomal poly ICLC.

In the efficacy study, groups of Balb/c mice (20 g) were pretreated intranasally with 2 doses of 20 μg of liposomal poly ICLC given at 48 hours apart. At 24 hours post drug treatment, these mice were intranasally challenged with either a low (one LD50) or a high (4 LD50) dose of avian influenza A virus. The survival rates of both virus control and treated animals were then monitored daily, and were determined at day 14 post infection.

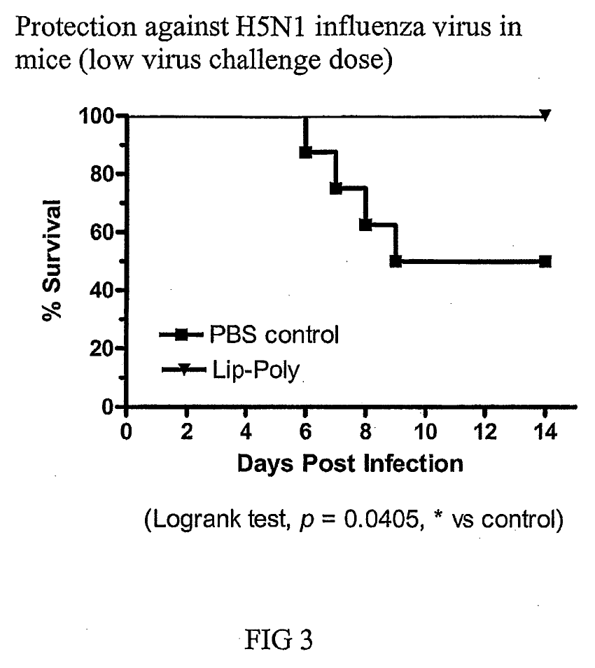

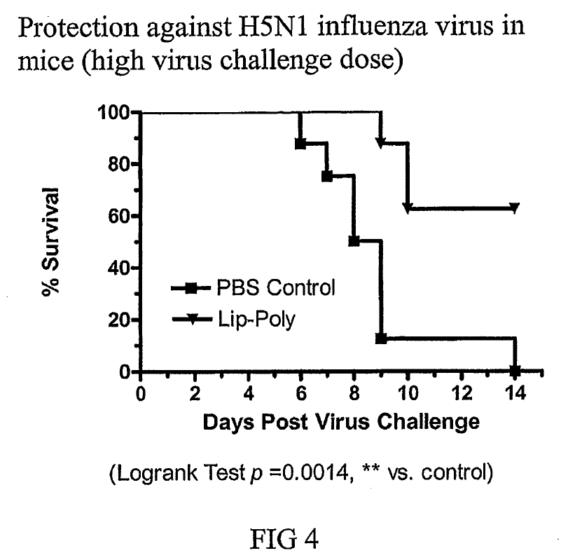

The efficacy of liposome-encapsulated poly ICLC for the prophylactic protection of mice against low and high virus challenge doses of avian H5N1 influenza virus in mice is shown in FIGS. 3 and 4, respectively. As shown in FIG. 3, control mice infected with either the low or high virus doses succumbed to the avian influenza infection starting at 5-6 days post infection. Half of the control mice infected with the low virus challenge dose died from the infection by day 9 post infection. In the high virus challenge dose, all mice died from the infection by day 14 post infection. In contrast, as shown in FIG. 4, liposome-encapsulated poly ICLC provided complete protection (100% survival) against the low virus challenge, and 63-75% protection against the high virus challenge. These levels of increased survival provided by liposomal poly ICLC were statistical significant (p<0.0402 vs control, p<0.0014 vs control, respectively, for the low and high virus doses). The mean survival times of mice pretreated with liposomal poly ICLC were found to have also increased as compared to control mice.

These results showed that liposome-encapsulated poly ICLC provides effective protection against avian influenza virus infections.

It should be noted that the dose of liposomal poly ICLC used in this H5N1 efficacy study was 20 μg per dose. However, other dosing regimen against the avian influenza virus in general, and more specifically, the H5N1 virus can also be used such as a range of 1-200 microgram per dose of which 1-100 microgram per dose is preferred. Of course, the dosing regimen can also be based on the weight of the subject to be treated in accordance with conventional understanding.

It is to be understood that the embodiments and variations shown and described herein are merely illustrative of the principles of this invention and that various modifications may be implemented by those skilled in the art without departing from the scope and spirit of the invention.

Also, wherever a range of values is provided in this disclosure, each intervening value and range, unless the context dictates otherwise, is encompassed within the invention. Further, it is understood that the invention includes, for each value, tenths of the lower limit indicated, unless the context clearly dictates otherwise. The invention also includes the upper and lower limit of the stated range, unless otherwise indicated. The upper and lower limits of smaller ranges (including intervening ranges) may independently be included in the smaller ranges. Where the stated range includes one or both of the limits, ranges excluding either or both of those included limits are also included in the invention.

In summary, liposomal poly ICLC provided protection against both seasonal and avian influenza viruses in experimental studies in animals. The observation that liposomal poly ICLC has been shown to be effective against various subtypes of influenza A viruses (H1N1, H3N2 and H5N1) establishes that it is particularly well suited to deal with the ever-changing, mutating viruses such as influenza viruses and in particular, avian influenza viruses. The experimental results presented herein collectively establishes that liposomal poly ICLC is an important drug that can be used to complement the existing arsenal of anti-influenza drugs and vaccines in the global fight against avian, pandemic and seasonal influenza.

While the invention has been described in detail with reference to preferred embodiments thereof, it will be apparent to one skilled in the art that various changes can be made, and equivalents employed, without departing from the scope of the invention. Each of the aforementioned documents is incorporated by reference herein in its entirety.

What is claimed is:

1. A method of prophylactically treating an avian influenza viral infection in a mammal comprising administering to said mammal a composition comprising polyriboinosinic and polyribocytidylic acids stabilized in poly-L-lysine and carboxymethylcellulose encapsulated within liposomes.

2. The method of claim 1, wherein the avian influenza viral infection is of a subtype H5N1.

3. The method of claim 1, wherein said liposomes are cationic liposomes comprising phosphatidylcholine, a cationic lipid and cholesterol.

4. The method of claim 2, wherein said liposomes are cationic liposomes comprising phosphatidylcholine, a cationic lipid and cholesterol.

5. The method of claim 3, wherein said cationic lipid is stearylamine.

6. The method of claim 4, wherein said cationic lipid is stearylamine.

7. The method of claim 3, wherein said phosphatidylcholine, cationic lipid and cholesterol are present in a molar ratio of 9:1:1, respectively.

8. The method of claim 4, wherein said phosphatidylcholine, cationic lipid and cholesterol are present in a molar ratio of 9:1:1, respectively.

9. The method of claim 5, wherein said phosphatidylcholine, stearylamine and cholesterol are present in a molar ratio of 9:1:1, respectively.

10. The method of claim 6, wherein said phosphatidylcholine, stearylamine and cholesterol are present in a molar ratio of 9:1:1, respectively.

11. The method of claim 1, wherein said administering comprises intranasal administration.

12. The method of claim 1, wherein said administering comprises intraperitoneal administration.

13. The method of claim 1, wherein said administering comprises intravenous administration.

14. The method of claim 2, wherein said administering comprises intranasal administration.

15. The method of claim 2, wherein said administering comprises intraperitoneal administration.

16. The method of claim 2, wherein said administering comprises intravenous administration.

17. The method of any claim 1, wherein said administering comprises administration by inhalation.

18. The method of claim 2, wherein said administering comprises administration by inhalation.
